(19) United States
(12) Patent Application Publication
(10) Pub. No.: US 2017/0296225 A1
(43) Pub. Date: Oct. 19, 2017

(54) STOMAL DILATOR

(71) Applicant: Dominic L. Diorio, Merchantville, NJ (US)

(72) Inventor: Dominic L. Diorio, Merchantville, NJ (US)

(21) Appl. No.: 15/130,868

(22) Filed: Apr. 15, 2016

(51) Int. Cl. A61B 17/34; A61B 17/00; A61J 15/00

(52) U.S. Cl. CPC A61B 17/3415; A61B 17/3494; A61B 17/3468; A61B 2017/0023; A61B 2017/3492; A61J 15/0015; A61B 2017/00818

(57) ABSTRACT

A stomal dilator comprising a proximal end; a distal end; a tapered dilation portion positioned between the proximal end and distal end having a first length and a varied diameter that progressively increases in size from the proximal end to the distal end; the proximal end including a filiform tip having a substantially constant diameter at second length, and the distal end including a handle portion having a substantially constant diameter third length; and a flange positioned on the distal end preventing the stomal dilator from entering into a patient.

### CROSS-REFERENCE TO RELATED APPLICATIONS

N/A

### BACKGROUND OF THE INVENTION

### Field of the Invention

The present invention relates to medical devices. More particularly, the present invention relates to a stomal dilator for use by patients having surgically created stomas.

### Background of the Invention

Many surgical procedures result in artificial openings, or stomas, being placed into a patient. For instance, if a patient was provided a feeding tube which was inserted through the abdominal wall into the stomach to supply the patient with essential nutrients in situations where the patient is unable to intake an adequate amount of nutrition by mouth. These feeding tubes travel from the outside of the body through the abdomen wall into the stomach, and are kept in place by an inflatable balloon. Occasionally, these feeding tubes, commonly called PEG (Percutaneous Endoscopic Gastrostomy) tubes are dislodged and come out of place. Often this occurs while a patient is sleeping, and it is not discovered until morning. If this happens the stoma immediately begins to close and within a period of 7 to 8 hours, it is difficult to replace the PEG tube unless the stoma is dilated. Currently, this is done using existing instruments that are cumbersome to use which are available in various sizes that are required to be exchanged for progressively larger sizes during the procedure. Furthermore, these instruments have to be thoroughly cleaned and sterilized before the next usage.

Consequently, there is a need for a single, self-contained, and disposable stomal dilator that may be easily inserted into a closed (stenosed) stoma while allowing a gradual progressive dilation.

### BRIEF SUMMARY OF THE INVENTION

In one embodiment of the present invention a stomal dilator is provided, comprising a proximal end; a distal end; a tapered dilation portion positioned between the proximal end and distal end having a first length; the proximal end including a filiform tip having a substantially constant diameter at second length, the distal end including a handle portion having a substantially constant diameter at a third length; and a flange positioned on the distal end preventing the stomal dilator from entering into a patient.

In one embodiment, the tapered dilation portion has a varied diameter that progressively increases in size from the proximal end to the distal end. In one embodiment, the varied diameter increases up to 22 French. In another embodiment, the first length is approximately 6 inches, the second and third length are approximately 1 inch correlating to the stomal dilator having a total length of 8 inches. In yet another embodiment, the first length is approximately 6 inches, the second length is approximately 2 inches, and third length is approximately 1 inch correlating to the stomal dilator having a total length of 9 inches. In one embodiment, the stomal dilator is constructed from a semi-rigid material, the material having strength necessary for dilation and flexibility to avoid injury to underlying organs of the patient. In another embodiment, the semi-rigid material is a medical grade plastic.

In another aspect to the invention, a stomal dilator is provided, comprising a proximal end; a distal end; a tapered dilation portion positioned between the proximal end and distal end having a first length and a plurality of dilation points that progressively increase in size from the proximal end to the distal end; the proximal end including a filiform tip having a substantially constant diameter at second length, and the distal end including a handle portion having a substantially constant diameter at a third length; and a flange positioned on the distal end preventing the stomal dilator from entering into a patient.

In one embodiment, the plurality of dilation points includes 16, 18, and 22 French. In another embodiment, the first length is approximately 5 to 6 inches. In one embodiment, the stomal dilator is constructed from a single integral unit. In yet another embodiment, the stomal dilator is disposable.

Such stated summary and advantages of the invention are only examples and should not be construed as limiting the present invention. These and other features, aspects, and advantages of the invention will be apparent to those skilled in the art from the following detailed description of preferred non-limiting exemplary embodiments, taken together with the drawings and the claims that follow.

### BRIEF DESCRIPTION OF THE SEVERAL VIEWS OF THE DRAWINGS

It is to be understood that the drawings are to be used for the purposes of exemplary illustration only and not as a definition of the limits of the invention. Throughout the disclosure, the word “exemplary” is used exclusively to mean “serving as an example, instance, or illustration.” Any embodiment described as “exemplary” is not necessarily to be construed as preferred or advantageous over other embodiments. Referring to the drawings in which like reference character(s) present corresponding part(s) throughout:

FIG. 1A is an exemplary diagram illustrating a stomal dilator according to an embodiment of the present invention.

FIG. 1B is an exemplary diagram illustrating the stomal dilator of FIG. 1A with dimensional annotations.

### DETAILED DESCRIPTION OF THE INVENTION

The detailed description set forth below in connection with the appended drawings is intended as a description of presently preferred embodiments of the invention and is not intended to represent the only forms in which the present invention may be constructed and or utilized.

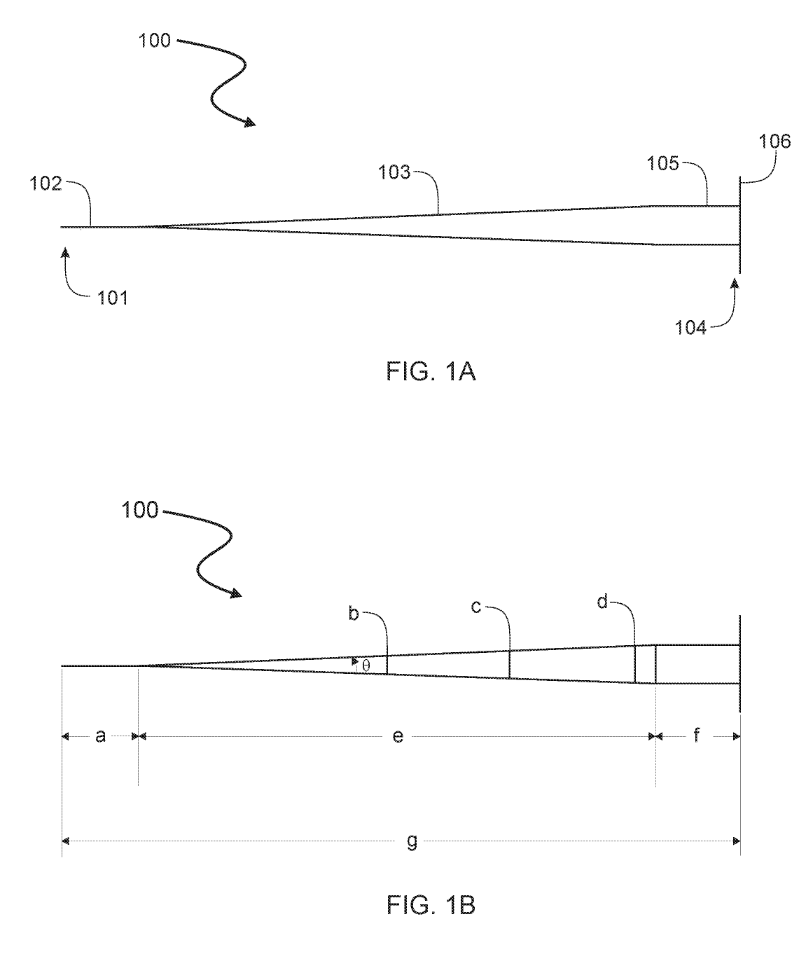

FIG. 1A is an exemplary diagram illustrating stoma dilator 100 according to an embodiment of the present invention. Now referring to FIG. 1A, the stomal dilator comprises a filiform tip 102 on a proximal end 101. The filiform tip is extremely narrow allowing the dilator o enter a stoma of a patient with minimal discomfort. The filiform tip is attached to a tapered dilation portion 103 having a progressively enlarging diameter towards a distal end 104. In operation, after the filiform tip has been inserted into the stoma the tapered dilation portion follows, allowing for a progressive dilation. The stomal dilator further comprises a handle portion 105 located at the distal end. In some embodiments, the handle portion be used to hold the stomal dilator during use, and is constructed of a substantially constant diameter. A flange 106 is attached to the distal end to prevent the stomal dilator from accidently entering an organ of the patient during use. For instance, if the stomal dilator slipped during use the flange would prevent the dilator from slipping into the stomach, bladder, or other organ. The flange may be constructed of any shape, including but not limited to square, rectangular, or circular.

FIG. 1B is an exemplary diagram illustrating the stomal dilator of FIG. 1A with dimensional annotations. In some embodiments, the filiform tip has a length (a) of approximately 1 to 2 inches, depending on the application and individual patient the stomal dilator is intended for. The filiform tip has an extremely narrow diameter which is substantially constant over the entirety of length (a). The tapered dilation portion comprises a plurality of dilation points (b, c, and d) corresponding to a number of progressively increasing diameters. In some embodiments, at dilation point (b) the diameter of the tapered dilation portion is 16 FR (French) on the French gauge scale corresponding to a diameter of 0.210 inches or 5,333 millimeters, In some embodiment, at dilation point (c) the diameter of the tapered dilation portion is 18 FR corresponding to a diameter of 0.236 inches or 6 millimeters. In some embodiment, at dilation point (d) the diameter of the tapered dilation portion is 22 FR corresponding to a diameter of 0.288 inches or 7.333 millimeters.

It is particularly advantageous of the present invention to provide a gradual increasing taper, corresponding to a taper angle (θ) of approximately 3 degrees, insuring precise dilation to any of the plurality of dilation points with minimal discomfort during use. The gradual increasing taper progressively increases in diameter over length (e) of the tapered dilation portion. In some embodiments, length (e) is approximately 5 to 6 inches, and preferably 6 inches. As previously mentioned, the handle portion has a substantially constant diameter over length (f), wherein length (f) is approximately 1 inch. The stomal dilator has a total length (g) which may vary based on individual lengths of the portions, but is mostly dependent on the length of the filiform tip (a). In some embodiments, total length (g) is approximately 8 inches when length (a) is 1 inch. In alternative embodiments, total length (g) is approximately 9 inches when length (a) is 2 inches.

In some embodiments, the stomal dilator is constructed from a semi-rigid material. The semi-rigid material having strength necessary for dilation and enough flexibility to avoid injury to underlying organs of the patient. In some embodiments, the semi-rigid material is a medical grade plastic, including but not limited to polyurethane, thermoplastic elastomer, or similar. It is particularly advantageous of the present invention to provide a disposable stomal dilator, consisting of a single integral unit that does not need to be progressively exchanged for different sized dilators or sent for sterilization. The stomal dilator minimizes the possibility of infections, as the stomal dilator is disposable and does not require sterilization between uses. The simplistic design and single integral unit construction allows for disposability.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention.

It should further be noted that throughout the entire disclosure, the labels such as left, right, front, back, top, bottom, forward, reverse, clockwise, counter clockwise, up, down, or other similar terms such as upper, lower, aft, fore, vertical, horizontal, oblique, proximal, distal, parallel, perpendicular, transverse, longitudinal, etc. have been used for convenience purposes only and are not intended to imply any particular fixed direction or orientation. Instead, they are used to reflect relative locations and/or directions/orientations between various portions of an object.

In addition, reference to “first,” “second,” “third,” and etc. members throughout the disclosure (and in particular, claims) are not used to show a serial or numerical limitation but instead are used to distinguish or identify the various members of the group.

In addition, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of,” “act of,” “operation of,” or “operational act of” in the claims herein is not intended to invoke the provisions of 35 U.S.C. 112, Paragraph 6.

What is claimed is:

1. A stomal dilator comprising:
a proximal end;
a distal end;
a tapered dilation portion positioned between the proximal end and distal end having a first length;
the proximal end including a filiform tip having a substantially constant diameter at second length, and the distal end including a handle portion having a substantially constant diameter at a third length; and
a flange positioned on the distal end preventing the stomal dilator from entering into a patient.

2. The stomal dilator of claim 1, wherein the tapered dilation portion has a varied diameter that progressively increases in size from the proximal end to the distal end.

3. The stomal dilator of claim 2, wherein the varied diameter increases up to 22 French.

4. The stomal dilator of claim 1, wherein the first length is approximately 6 inches, the second and third length are approximately 1 inch correlating to the stomal dilator having a total length of 8 inches.

5. The stomal dilator of claim 1, wherein the first length is approximately 6 inches, the second length is approximately 2 inches, and third length is approximately 1 inch correlating to the stomal dilator having a total length of 9 inches.

6. The stomal dilator of claim 1, wherein the stomal dilator is constructed from a semi-rigid material, the material having a strength necessary for dilation and a flexibility to avoid injury to underlying organs of the patient.

7. The stomal dilator of claim 6, wherein the semi-rigid material is a medical grade plastic.

8. A stomal dilator comprising:
a proximal end;
a distal end;
a tapered dilation portion positioned between the proximal end and distal end having a first length and a plurality of dilation points that progressively increase in size from the proximal end to the distal end;
the proximal end including a filiform tip having a substantially constant diameter at second length, and the distal end including a handle portion having a substantially constant diameter third length; and
a flange positioned on the distal end preventing the stomal dilator from entering into a patient.

9. The stomal dilator of claim 8, wherein the plurality of dilation points includes 16, 18, and 22 French.

10. The stomal dilator of claim 9, wherein the first length is approximately 5 to 6 inches.

11. The stomal dilator of claim 9, wherein the stomal dilator is constructed from a single integral unit.

12. The stomal dilator of claim 11, wherein the stomal dilator is disposable.
